(19) United States
(12) Patent Application Publication
(10) Pub. No.: US 2004/0117128 A1
(43) Pub. Date: Jun. 17, 2004

(54) Methods, computer software products and systems for gene expression cluster analysis

(75) Inventor: Jill Cheng, Burlingame, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/317,512

(22) Filed: Dec. 11, 2002

(51) Int. Cl. G06F 19/00; G01N 33/48; G01N 33/50

(52) U.S. Cl. 702/20

(57) ABSTRACT

In some embodiment of the invention, methods are provided to classify genes based upon biological knowledge. The methods are useful for analyzing biological data such as gene expression data.

### RELATED APPLICATIONS

This application claims the priority of U.S. Provisional Application Serial No. 60/297,210.

This application is related to U.S. patent application Ser. Nos. 10/026,110, 10/256,938 and ______, attorney docket 3539, titled “Statistical Analysis for Gene Ontology”, filed on Dec. 3, 2002 and U.S. patent application Ser. No. ______ Docket Number 3546, filed concurrently herewith. The cited applications are incorporated herein by reference.

### BACKGROUND OF THE INVENTION

This invention is related to bioinformatics, computer software and computer systems.

As a result of the successful development of high-throughput gene expression analysis technologies, most notably the high-density microarray technology, massive amounts of data are being generated. Establishing biological interpretation from these large data sets is a challenging and often rate-limiting step in data analysis. Therefore, there is a great need in the art for methods, computer software and systems for analyzing gene expression data to derive biological interpretation.

### SUMMARY OF THE INVENTION

In one aspect of the invention, two computer implemented methods are provided for genes clustering based on biological annotations. The exemplary methods of the invention employ the directed acyclic graph (digraph) structure of Gene Ontology (GO) and annotations using GO terms. GO is a dynamic controlled vocabulary for molecular biology, GO terms are structured as a digraph; the biological relationship between GO terms is represented by the edges in the digraph. The relationships between genes can be projected by the relationships between the GO terms with which genes are annotated.

In some embodiments, the computer implemented methods automatically cluster genes based upon existing knowledge represented in GO annotations. In some other embodiments, a novel similarity matrix that is generated by combining a knowledge-based matrix and an expression-profiling matrix. the resulting clusters from these algorithms are ranked by statistical methods such as Fisher Exact Test.

In another aspect of the invention, computer systems and software products are provided to perform the methods of the invention. A computer software product typically includes a computer medium with computer software codes that executes the methods of the invention in a computer system.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 shows the relationship between GO annotation terms.

FIG. 2 illustrates the GO digraph.

FIG. 3 shows the structure of an annotation database that is useful in some embodiments of the invention.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

1. General

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W.H. Freeman Pub., New York, N.Y. and Berg et al. (2002) Biochemistry, 5th Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays which are also described.

Nucleic acid arrays that are useful in the present invention include those that are commercially available from Affymetrix (Santa Clara, Calif.) under the brand name GeneChip®. Example arrays are shown on the website at affymetrix.com. The present invention also contemplates many uses for polymers attached to solid substrates. These uses include gene expression monitoring, profiling, library screening, genotyping and diagnostics. Gene expression monitoring, and profiling methods are shown in U.S. Pat. Nos. 5,800,992, 6,013,449, 6,020,135, 6,033,860, 6,040,138, 6,177,248 and 6,309,822. Genotyping and uses therefore are shown in U.S. S No. 60/319,253, U.S. Ser. No. 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598, which are incorporated herein by reference for all purposes.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80:1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouclette and Bzevanis Bioinformatics: A Practical Guide for Analysis of Gene and Proteins (Wiley & Sons, Inc., 2nd ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170, which are incorporated herein by reference.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

II. Glossary

The following terms are intended to have the following general meanings as used herein.

Nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

A nucleic acid library or array is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases (see, e.g., U.S. Pat. No. 6,156,501, incorporated herein by reference). The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids. See, e.g., U.S. Pat. No. 5,143,854.

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point (Tm) fro the specific sequence at a defined ionic strength and pH. The Tm is the temperature (under defined ionic strength, pH and nucleic acid composition) at which 50% of the probes complementary to the target sequence hybridize to the target sequence at equilibrium.

Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2nd Ed. Cold Spring Harbor Press (1989) and Anderson “Nucleic Acid Hybridization” 1st Ed., BIOS Scientific Publishers Limited (1999), which are hereby incorporated by reference in its entirety for all purposes above.

Hybridization probes are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501.

Probe: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

Effective amount refers to an amount sufficient to induce a desired result. mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges maybe from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful. See, e.g., Dong et al., Genome Research 11, 1418 (2001), in U.S. Pat. Nos. 6,361,947, 6,391,592, incorporated herein by reference.

A primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

A genome is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments represents the whole or a portion of a genome. Frequently, a genomic libarry is a collection of clones made from a set of randomly generated, sometimes overlapping DNA fragments representing the entire genome or a portion of the genome of an organism.

An allele refers to one specific form of a genetic sequence (such as a gene) within a cell or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”.

At each autosomal specific chromosomal location or “locus” an individual possesses two alleles, one inherited from the father and one from the mother. An individual is “heterozygous” at a locus if it has two different alleles at that locus. An individual is “homozygous” at a locus if it has two identical alleles at that locus.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Single nucleotide polymorphism (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Genotyping refers to the determination of the genetic information an individual carries at one or more positions in the genome. For example, genotyping may comprise the determination of which allele or alleles an individual carries for a single SNP or the determination of which allele or alleles an individual carries for a plurality of SNPs. A genotype may be the identity of the alleles present in an individual at one or more polymorphic sites.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles. A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be detected to indicate susceptibility to the disease in circumstances in which the gene Y may not have been identified or may not be readily detectable.

III. Pair-Wise Similarity Measures Between GO Terms

In one aspect of the invention, methods, computer software products and systems are provided for assigning pair-wise similarity measures between annotation terms for genes. The pair wise similarity measures are particularly useful for clustering related genes based upon biological knowledge and for analyzing gene expression results. While the methods, computer software and products are illustrated using the GO terms as examples, one of skill in the art would appreciate that they are also useful for other annotation systems.

Biological knowledge, as used herein, refers to information that describes the function (e.g., at molecular, cellular and system levels), structure, pathological roles, toxicological implications, etc. Various biological knowledge systems can be used for the methods of the invention. In preferred embodiments, the annotation systems used by the Gene Ontology (GO) Consortium or similar systems are employed. GO is a dynamic controlled vocabulary for molecular biology which can be applied to all organisms as knowledge of gene is accumulating and changing, it is developed and maintained by Gene Ontology™ Consortium (Gene Ontology: tool for the unification of biology. The Gene Ontology Consortium (2000) Nature Genet. 25: 25-29). Currently, there are three categories of GO terms: biological processes, molecular function, and cellular component.

A gene can be annotated with several GO terms. For example, the Degenerin gene is annotated with “peripheral nervous system development”, “monovalent inorganic cation transport”, “central nervous system development”, and “synaptic transmission” for biological process, “amiloride-sensitive sodium channel” for molecular function, and “integral plasma membrane protein” for cellular component. Gene annotations using GO terms provide an excellent resource for summarized knowledge on each gene. Genes with similar biological property are annotated with the same or similar GO terms and thus can be easily identified.

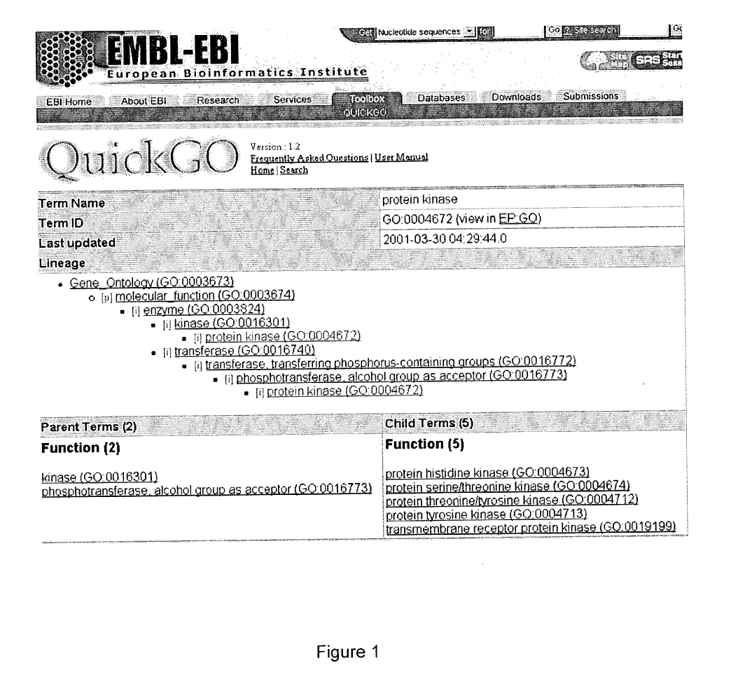

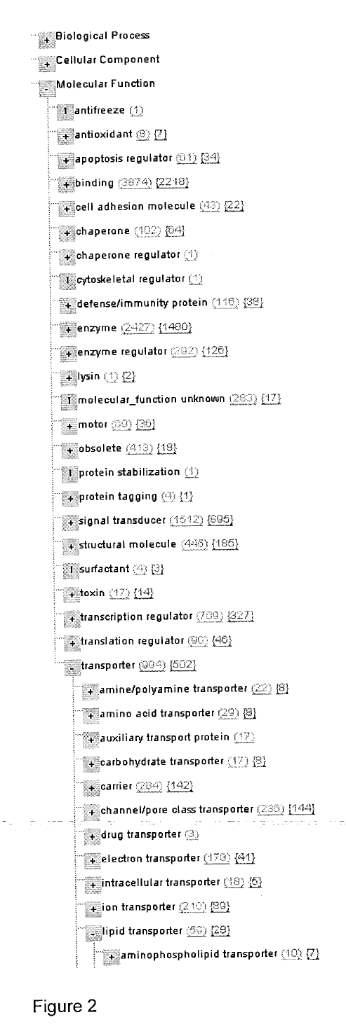

GO terms are structured as a diagraph (see a graphic illustration, FIGS. 1 and 2), which means one term may be a child or a parent to other terms, one term may have multiple parents or multiple children. This relationship is often referred to as multiple inheritance in this specification. Considering each GO term as a node in the digraph, two connected nodes defines an edge. A collection of connected edges defines a “path”. For instance, “protein kinase” has two parents: “kinase” and “phosphotransferase”, and five children: “protein histidine kinase”, “protein serine/theronine kinase”, “protein threonine/tyrosine kinase”, “protein tyrosine kinase”, and transmembrane receptor protein kinase” (See, FIG. 1). The closer a GO term is to the root of the digraph, the more general the biological classification is. The relative position of two nodes in the digraph reflects the biological distance between the two terms. For example, protein tyrosine kinase and protein histidine kinase are both child nodes of protein kinases, they share higher biological relevance then with another term that has a different parent node, for example amiloride-sensitive sodium channel. Genes annotated with protein tyrosine kinase and protein histidine kinase, respectively, are functionally similar since they both fall in the category of protein kinase. Thus, the more common ancestors two GO terms share in their paths, the more similar they are biologically.

Current analytical methods involving GO primarily use GO terms as a keyword system where genes annotated with the same GO terms are recognized. This approach has not taken the full advantage of GO since relationships between GO terms are not captured, in fact, genes annotated with different GO terms may be closer then genes annotated with the same GO term, depending on the geographic positions of the GO terms. For example, two proteins annotated with JUN_kinase and SAP-kinase respectively may have higher similarity to each other based upon existing facts then the two genes both annotated with the same term phosphotransferase, because phosphotransferase is a more general term and is closer to the root. Thus, using GO annotations without considering the overall digraph structure can be misleading. Tracing the topological locations of GO terms in the GO digraph allows one to assess the biological relationship among terms accurately and so the biological similarity between genes can be assessed more sensitively.

In some embodiments, the methods of the invention transform the biological knowledge captured in GO into a numeric form based upon the digraph structure. Pair wise similarity matrix between GO terms is generated based upon the relative positions of GO terms in GO paths. This matrix is used as the similarity measurements between two genes based upon gene annotations using GO terms. Since one gene may have multiple GO annotations, a greedy approach may be used by taking the highest scoring pair of GO terms for two genes to assign the pair wise similarity score.

In Gene Ontology, each edge represents the relationship of “is a” or “is a part of”, meaning that a child node is either a part of the parent or is a more specific example of the parent term. So the closer a term is to the root, the more general is its biological classification. Similar to the phylogeny classifications; the deeper the edge in a path, the finer or more resolved the classification. For example, “molecular function” has child nodes including “transcription regulator” and “transporter”, “transporter” has child nodes including “ion transporter” and “lipid transporter”; “lipid transporter” has child nodes including “fatty acid transporter” and “sterol transporter” (See, FIG. 2). The similarity in-between two nodes which share a 3-edge partial path “molecular function—transporter-lipid transporter-sterol transporter” is higher then two nodes sharing a 2-edge partial path “molecular function transporter-lipid transporter”. And since the classification gets finer toward the leaf, the edge “lipid transporter-sterol transporter” has less added similarity value then “transporter-lipid transporter”.

A weighting factor (wt) may be assigned to each edge as a function of the level in a path. The closer to the root the higher the weigh is. The level starts at the root, level=n=0. The weight for a partial path consists of p edges is Wₚ. Because the biological classification becomes less significant towards the leaf, the weight may be chosen to be less then one so that the sum will reach convergence (C) when the length of a path reaches infinity.

$$\begin{matrix}{W}_{p} = {\sum }_{n = 0}^{p} {( w t )}^{n} \\ C = Convergence = {\sum }_{n = 0}^{∞} {( w t )}^{n}\end{matrix}$$

The degree of similarity between two nodes is correlated with the number of edges they have in common in the paths leading to these nodes, represented by the length of the shared partial path. The longer the shared partial path the higher the degree of biological similarity is.

The number of edges below the point of divergence, that is, below the lowest level common ancestor, does not contribute further to the biological similarity. Only the number of edges that are shared is significant. By adjusting the weighting factor, one can manipulate the stringency of similarity scaling.

Because of multiple inheritances, one node may reside in multiple paths. A greedy approach may be taken to select the longest common partial path in order to maximize the ability to capture similarity between nodes.

Based on extensive review of the GO graph, it was observed that shorter paths usually have more coarse classification than longer paths; that is, an edge in a short path tends to have slightly more biological significance then an edge in a long path. Therefore, a partial normalization scheme may be applied to factor in the unevenness of GO digraph.

A partial normalization factor (Nfₚ) is derived as follows. The average length from for all paths that go through the shared partial paths are calculated (p), the weight for a hypothetical path with p edges is calculated (Wₚ). To do a partial normalization, Wₚ is normalized to Wₚ′ which is the mean of Wₚ and C with normalization Nfₚ, thus Nfₚ is derived by dividing Wₚ′ with Wₚ.

$$\begin{matrix}{W}_{{p}^{′}} = \frac{{W}_{p} + C}{2} \\ N {f}_{p} = \frac{{W}_{{p}^{′}}}{{W}_{p}}\end{matrix}$$

The value for the shared partial path with m edges (Wₘ) can then be calculated with partial normalized by applying Nfₚ.

$${W}_{m} = N {f}_{p} {\sum }_{n = 0}^{m} {( W t )}^{n}$$

This value is the similarity score for each GO term pair. GO term share 0 common edge will have a 0 sore. All pair wise scores among GO terms together form the GO similarity matrix.

Computer software for calculating the similarity measures may be written in any suitable languages. The computer software codes can be created to execute the methods described above. The software codes may be stored in a suitable computer readable medium. Many computer systems are suitable for executing the methods for calculating similarity measures between annotation terms.

IV. Biological Knowledge Database

In another aspect of the invention, a biological knowledge database is provided. Biological knowledge is collected from public sources such as Locuslink, Unigene, SwissTrEMBL, etc, and organized into a relational database following the concept of the central dogma of molecular biology. The database entities are modeled after biological entities and the relationship between them. For instance, one genetic locus can produce one or more transcripts, one transcript can generate zero to many proteins, and one protein may has zero or many domains, table locus, transcript, protein, and domain were designed. Following these rules, table locus is linked to table transcript with a one-to-many relationship, table transcript is linked to table protein with a zero-to-many relationship, and table protein is linked to table domain with a zero-to-many relationship.

Two tables are designed to represent the digraph structure of GO, one table for GO terms (nodes) and one table for the parent-child relationship between two terms (edge). Since one gene can have many GO term annotations and one GO term can be used in the annotations for many genes, the GO annotations for specific genes are stored in the join table in between locus table and GO-terms table. Tables holding Affymetrix probesets (www.netaffx.com, Affymetrix, Inc., Santa Clara, Calif.) are associated with the locus table through Genbank accession tables. Through the locus table, the connection between Affymetrix probesets and GO annotations can be established.

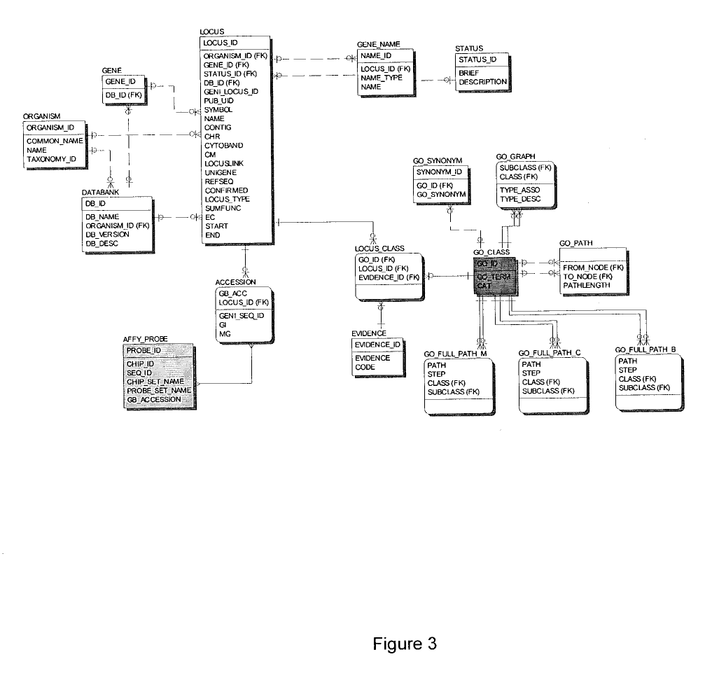

In order to eliminate the need for real-time path enumeration, graph algorithm may be used to resolve all possible full or partial paths for every given GO node. The results from path enumeration are stored into several derived tables in the database. Since the terms within each of the three GO categories are unique to the categories, each GO category is treated as an independent digraph and separate tables are created to hold the path enumeration results. Thus, the root for each sub-digraph is “molecular function”, “biological process”, or “cellular component”. Querying derived path tables allows rapid calculation of similarity between two nodes based on the maximum length of shared partial paths in-between two given nodes. See FIG. 3 for a view of the data model for the Knowledge Integration Database.

V. Gene Clustering for Gene Expression Data Analysis

The typical process for gene expression profile analysis involves clustering genes according to expression profiles, and then interpreting or validating the clusters according to functional annotations of the clustered genes (See, e.g., U.S. Pat. No. 6,303,301, incorporated herein by reference). The most popular statistical clustering methods include K-means, Self-Organized Maps (SOM), etc. Each cluster generated by a statistical method requires further verification by a domain expert to determine whether or not it makes biological sense and whether or not the genes found in the same cluster also share similar functionality.

Because it is inefficient to manually derive biological interpretations from a large number of genes, one common practice today is to apply stringent statistical conditions to weed out noisy leads and narrow down the number of leads. Often, the statistical approach sacrifices the sensitivity in detecting results that are biologically significant but less obvious.

Furthermore, different statistical clustering methods usually give rise to different results; it is prone to arbitrary downstream interpretations due to analysis methods or parameters used.

In one aspect of the invention, the gene clustering analyses are improved by methods which incorporate biological knowledge directly into the analysis methodology, and which provides an automatic solution to facilitate verification and interpretation of expression results and alleviate most of the manual effort.

The exemplary methods of the invention employ the directed acyclic graph (digraph) structure of Gene Ontology (GO) and annotations using GO terms.

In some embodiments, a GO cluster algorithm is employed in computer implemented methods to automatically cluster genes based upon existing knowledge. In some other embodiments, a GO-guided cluster algorithm is used to cluster genes based upon a novel similarity matrix that is generated by combining a knowledge-based matrix and an expression-profiling matrix.

The Go Cluster Algorithm clusters a list of genes based upon their biological annotations. The gene list can be statistical clustering results or simply a list of up-regulated genes.

The exemplary algorithm clusters genes with similarity in their biological functions or processes within a threshold value, and report the significant subgroups labeled with GO terms. This process allows rapid evaluation of statistical clustering results and automatic generation of biological interpretation.

It is well known that genes with similar expression profiles are often involved in the same biological process, but genes involved in same biological process, however, do not all have the same expression patterns. In one embodiment, a filter, such as a filter that selects genes with varying expression level along a time course, following by knowledge based analysis is used to identify trends in gene regulation under various experimental conditions. Such a filter alone, which imposes no constrain on the similarity on expression profile of the target genes, typically generates a larger gene list and is very difficult for downstream analysis.

For GO clustering, a unique set of GO terms used in the annotations for a list of genes are pooled and clustered using GO similarity matrix. Each identified GO cluster is named with the GO term that is the lowest level common ancestor. Genes annotated with terms in the same cluster are pulled together to form a gene cluster. Since one gene may have multiple GO term annotations, one gene may be classified into multiple clusters independently.

Multiple-inheritance is an important characteristic in biology, and it is prominent in GO digraph. Because proteins often have multiple domains and multiple functions, it is possible that gene A and gene B share functional similarity through one protein domain, and gene C and gene B share functional similarity through another protein domain, and gene A and gene C share no functional similarity. Such property is captured by the Multiple-inheritance of GO digraph. For example, if node A and node B are close, node B and node C are also close, it is possible that node A and node C are very distant. Because node B may have 2 parent nodes and each separately account for the similarity measure with node B and with node C. Thus, it is important for a clustering algorithm to embrace this nature by allowing one term to be clustered into multiple clusters independently.

To accommodate the multi-function and multi-domain characteristic of proteins, the clique detecting technique is used for clustering, which allows one gene to be classified into multiple clusters independently. For a group of pair wise measurements, a cliques is a subgroup where all pair wise measures within the clique are all above a threshold. For example, if AB=10, AC=8, BC=2<-- less then 4, AD=12, CD=9 BD=9, a clique would be ABD when a threshold of 4 is applied, C is not in the clique because for C to be in the clique, AC, BC, CD all have to be above the threshold but BC is not. A computer implemented algorithm may be used for searching the cliques among a list of pair wise measurements. For a description of clique finding algorithm, see, e.g., Introduction to Algorithms, Second Edition, by Thomas H. Cormen (Editor), Charles E. Leiserson, Ronald L. Rivest, MIT Press; ISBN: 0262032937; 2nd edition (Sep. 1, 2001), incorporated herein by reference.

For GO-guided clustering, Euclidean distances between genes are generated based upon expression profiling data and then be converted to a similarity score with value between 0 and 5 by subtraction from value 5. This is the formation of expression profiling based similarity matrix. Combining this matrix with GO similarity matrix generates a new similarity matrix. One way to combine the two matrix is by summing at a certain ratio. The combined matrix reflects a compounded degree of similarity in both expression patterns and biological annotations. This combined matrix is then clustered using the clique-finding clustering algorithm to generate gene clusters.

Whether or not a functional cluster identified from a given gene list represents true enrichment for gene in this functional category by experimental conditions is important for biological interpretation. This can be assessed by the probability that the expression profiles for this number of genes in this GO class would be significantly altered with the experimental treatment solely by chance. In one embodiment, Fisher Exact Test is used to rank each gene cluster and the highest ranked clusters are used for biological interpretation.

### VI. EXAMPLE

Transgenic Myeloid Progenitor cells (MPRO) cells transgenic for dominant negative Retinoic Acid (RA) receptor were induced to differentiate into Neutrophil with high doses of RA. Gene expression at 0, 1, 2, 4, and 8 hours post RA induction was analyzed with Affymetrix GeneChip® U74A probe array. Four replicates were used at each of the 5 time points, for a total of 20 arrays. Signal values were generated and scaled with Microarray Analysis Suite 5.0 (Affymetrix, Santa Clara, Calif.). Each array was further normalized by adding a small value to these Signals so that the kurtosis of the population of log transformed Signals was equal the value for a normal distribution. These log transformed values were then used in subsequent analysis.

In the case where multiple probesets are designed for the same gene, only one probeset was kept for the subsequent analysis to avoid redundancy. Probesets showing change across the 5 time points was determined as follows. (1) The pooled standard deviation at each time point was computed using all probesets. (2) Each log signal value was divided by the pooled standard deviation corresponding to the time point at which the measurement was taken. (3) An ANOVA was performed for each probeset using its 20 normalized measurements from step (2), resulting in an F-statistic score for each probeset. (4) All probesets were ranked according to this statistic, and the top 80 were kept for input to the next stage of processing.

The 80 probesets showing maximal change across the time points collected. The 20 values (5 time points in 4 replica) for each probeset were pre-processed as follows. First, the 4 replicate values at each time point were averaged. Second, the 5 averaged values for each probeset were transformed to have zero mean and unit variance. This data set was then analyzed with various conventional cluster algorithms including Hierarchical, K-means, SOM, and GO clustering and GO-guided clustering as well.

Genes showing changes in expression levels during the time course were collected, and analyzed by GO clustering or GO-guided clustering. Major functional categories showing changes during the course of leukocyte differentiation were identified by GO clustering algorithm and ranked by Fisher exact test. Sub-populations of genes sharing functional similarity were isolated within a cluster of co-expressed genes automatically. Genes clustering based upon expression pattern by K-means, SOM, or hierarchical cluster algorithms were compared with GO-guided clustering. It was demonstrated that both algorithms improve the efficiency and quality of expression data analysis.

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

What is claimed is:

1. A computer implemented method for gene expression analysis comprising:
Obtaining a unique set of annotation terms for a plurality of interested genes;
Performing a cluster analysis to obtain clusters for the annotation terms; and
Assigning interested genes into the clusters according to their annotation terms.

2. The method of claim 1 wherein the gene annotation terms are GO terms.

3. The method of claim 2 wherein the cluster analysis is based upon pair wise similarity measures between the GO terms.

4. The method of claim 3 wherein at least one interested gene is assigned to a plurality of clusters.

5. The method of claim 3 wherein the cluster analysis is performed with a clique finding algorithm.

6. The method of claim 3 wherein the pair wise similarity measures are determined according to the GO digraph paths.

7. The method of claim 6 wherein each of the pair wise similarity measures is calculated based upon the length of partial path shared by two annotation terms.

8. The method of claim 7 wherein a weighing factor is assigned to each edge as a function of the level in a path.

9. The method of claim 8 wherein the stringency of similarity scaling may be adjusted by adjusting the weighting factor.

10. The method of claim 7 wherein a greedy method is used to select the longest common partial path when an annotation term is in multiple paths.

11. A computer implemented method for gene expression analysis comprising:
Calculating Euclidean distances between a plurality of genes based upon gene expression profiling data;
Combining the Euclidean distances with gene annotation similarity matrix to generate a gene similarity matrix; and
Performing a cluster analysis on the gene similarity matrix to assign genes into clusters.

12. The method of claim 11 wherein the gene annotation similarity matrix contains pair wise similarity measures between the GO terms.

13. The method of claim 12 wherein at least one interested gene is assigned to a plurality of clusters.

14. The method of claim 13 wherein the cluster analysis is performed with a clique finding algorithm.

15. The method of claim 12 wherein the pair wise similarity measures are determined according to the GO digraph paths.

16. The method of claim 15 wherein each of the pair wise similarity measures is calculated based upon the length of partial path shared by two annotation terms.

17. The method of claim 16 wherein a weighing factor is assigned to each edge as a function of the level in a path.

18. The method of claim 18 wherein the stringency of similarity scaling may be adjusted by adjusting the weighting factor.

19. The method of claim 18 wherein a greedy methods is used to select the longest common partial path when an annotation term is in multiple paths.

20. The method of claim 11 wherein the Euclidean distances are converted to similarity scores by subtraction from 5.

21. The method of claim 20 wherein the combing comprises summing the Euclidean distances with GO similarity matrix at a ratio to generate the gene similarity matrix.

22. The method of claim 21 wherein Fisher Exact Test is used to rank each cluster.

23. The method of claim 22 wherein the highest ranked clusters are used for biological interpretation.

24. A computer readable medium having software modules for performing the method of: Obtaining a unique set of annotation terms for a plurality of interested genes; Performing a cluster analysis to obtain clusters for the annotation terms; and Assigning interested genes into the clusters according to their annotation terms.

25. The computer readable medium of claim 24 wherein the gene annotation terms are GO terms.

26. The computer readable medium of claim 25 wherein the cluster analysis is based upon pair wise similarity measures between the GO terms.

27. The computer readable medium of claim 26 wherein at least one interested gene is assigned to a plurality of clusters.

28. The computer readable medium of claim 26 wherein the cluster analysis is performed with a clique finding algorithm.

29. The computer readable medium of claim 26 wherein the pair wise similarity measures are determined according to the GO digraph paths.

30. The computer readable medium of claim 29 wherein each of the pair wise similarity measures is calculated based upon the length of partial path shared by two annotation terms.

31. The computer readable medium of claim 30 wherein a weighing factor is assigned to each edge as a function of the level in a path.

32. The computer readable medium of claim 31 wherein the stringency of similarity scaling may be adjusted by adjusting the weighting factor.

33. The computer readable medium of claim 29 wherein a greedy method is used to select the longest common partial path when an annotation term is in multiple paths.

34. A computer readable medium having software modules for performing the method of:
Calculating Euclidean distances between a plurality of genes based upon gene expression profiling data;
Combining the Euclidean distances with gene annotation similarity matrix to generate a gene similarity matrix; and
Performing a cluster analysis on the gene similarity matrix to assign genes into clusters.

35. The computer readable medium of claim 34 wherein the gene annotation similarity matrix contains pair wise similarity measures between the GO terms.

36. The computer readable medium of claim 35 wherein at least one interested gene is assigned to a plurality of clusters.

37. The computer readable medium of claim 36 wherein the cluster analysis is performed with a clique finding algorithm.

38. The computer readable medium of claim 37 wherein the pair wise similarity measures are determined according to the GO digraph paths.

39. The computer readable medium of claim 38 wherein each of the pair wise similarity measures is calculated based upon the length of partial path shared by two annotation terms.

40. The computer readable medium of claim 39 wherein a weighing factor is assigned to each edge as a function of the level in a path.

41. The computer readable medium of claim 40 wherein the stringency of similarity scaling may be adjusted by adjusting the weighting factor.

42. The computer readable medium of claim 40 wherein a greedy methods is used to select the longest common partial path when an annotation term is in multiple paths.

43. The computer readable medium of claim 40 wherein the Euclidean distances are converted to similarity scores by subtraction from 5.

44. The computer readable medium of claim 43 wherein the combing comprises summing the Euclidean distances with GO similarity matrix at a ratio to generate the gene similarity matrix.

45. The computer readable medium of claim 44 wherein Fisher Exact Test is used to rank each cluster.

46. The computer readable medium of claim 45 wherein the highest ranked clusters are used for biological interpretation.
